(19) United States
(12) Patent Application Publication
(10) Pub. No.: US 2023/0301579 A1
(43) Pub. Date: Sep. 28, 2023

(54) METHOD FOR THE MANAGEMENT OF ORAL THERAPY IN PARKINSON'S DISEASE

(71) Applicant: Brain Innovations, S.r.l., Roma (IT)

(72) Inventor: Lazzaro DI BIASE, Roma (IT)

(21) Appl. No.: 18/025,092

(22) PCT Filed: May 25, 2021
(86) PCT No.: PCT/IT2021/050161

§ 371 Date: Mar. 7, 2023

(30) Foreign Application Priority Data
Sep. 10, 2020 (IT) 102020000021385

(51) Int. Cl. A61B 5/00; A61B 5/145; A61B 5/11

(52) U.S. Cl. CPC A61B 5/4082; A61B 5/4803; A61B 5/004; A61B 5/14546; A61B 5/14514; A61B 5/4839; A61B 5/6802; A61B 5/1101; A61B 2562/0219; A61B 2562/0223; A61B 5/14528

(57) ABSTRACT

A method and a system are described, for managing a mouth therapy in the Parkinson's disease, through the analysis of the motion status of patients, wherein the motion status can be in one of the following phases: OFF phase, wherein Parkinson's symptoms, such as rigidity, tremor and bradykinesia, emerge; ON phase wherein the symptoms markedly improve; DIS phase wherein involuntary movements emerge, called dyskinesias; as detectors of the symptoms the following parameters are used: voice analysis; face analysis; tremor analysis; movement analysis; levodopa and metabolite levels of monoamines in the sub-cutaneous interstitial liquid of the patients; an algorithm being provided, which provides to the patient the daily therapeutic scheme with its relates administration dosages and inter-times, computed by using the parameters as detectors.

The present invention relates to a method for supporting the management of oral therapy in Parkinson's disease.

The invention is part of the technical field of medicine, in particular neurology.

Parkinson's disease is the most frequent neurodegenerative disease after Alzheimer's disease. The diagnosis of Parkinson's disease is essentially clinical and currently based on the objective and anamnestic identification of the characteristic signs and symptoms of the disease and on the exclusion of any atypical symptoms.

Patients with Parkinson's disease can experience fluctuations in their motor status during the day going from an OFF phase, in which parkinsonian symptoms such as rigidity, tremor and bradykinesia emerge, to an ON phase in which these symptoms improve significantly. marked, with a phase characterized by involuntary movements called dyskinesias (DIS). These fluctuations are due to fluctuations in dopamine levels in the central nervous system. The drug used to improve the motor symptoms of Parkinson's disease is levodopa, which is converted in the central nervous system to dopamine. Motor fluctuations consist of an initial benefit on motor symptoms after the administration of a dose of levodopa (time in motor ON) followed by a return of parkinsonian symptoms, such as slowing of movements, rigidity or tremor (time in motor OFF) that lasts until at the beginning of the benefit of the next dose. These fluctuations lead to a continuous oscillation of the patient's motor performance during the day, with an alternation of motor ON states, in which parkinsonian symptoms are well controlled by motor OFF therapy, in which parkinsonian symptoms re-emerge. Dyskinesias, on the other hand, are involuntary movements induced by levodopa. An increase in Levodopa doses can reduce motor OFF times, but tends to increase dyskinesias, while a reduction in levodopa doses can reduce dyskinesias, but tends to increase motor OFF time. In these patients, it may be difficult to draw up a daily schedule of oral levodopa doses to be taken that is able to satisfactorily control total motor OFF time without inducing dyskinesias.

Methods of adaptive therapy are known that modulate the therapy conveyed through deep brain stimulation (DBS—Deep Brain Stimulation).

Conventional DBS consists of the surgical implantation of two electrodes within a specific area of the brain (generally subthalamic nucleus or internal pale globe). Electrodes which are then connected with thin cables to a small stimulator placed under the skin near the collarbone.

However, this method has shown some limitations. First of all, the stimulation is constantly delivered to the patient's brain, with an intensity, therefore, not always suited to his needs.

This occurs because, in the advanced stages, the motor symptoms of Parkinson's disease are fluctuating. In a few seconds we pass from motor block to very disabling involuntary movements.

To overcome this limitation, an adaptive DBS has been developed, i.e. a stimulation that adapts automatically and in real time to the patient's clinical status. In other words, the electrical stimulus varies according to the brain activity detected moment by moment. In this way it is always calibrated to the patient's status.

However, adaptive DBS uses the measurement of brain activity as the only parameter to modulate the therapy and this fact does not allow the patient's motor condition to be correctly identified. It is also highly invasive, as it requires the insertion of electrodes into the subthalamic nucleus or the internal pale globe.

The purpose of the present invention is to provide a method, and a system for implementing said method, respectively in accordance with claims 1 and 2, to modulate oral therapy using the data of the initial therapeutic scheme as feedback.

The method for the management of oral therapy in Parkinson's disease, called management taking place through the analysis of the motor state of patients with said Parkinson's disease, in which said motor state may be in one of the following phases:
OFF phase, in which parkinsonian symptoms such as rigidity, tremor and bradykinesia emerge;
ON phase, in which said symptoms improve markedly;
DIS phase, in which involuntary movements defined as dyskinesias emerge;
   is characterized by using the following parameters as detectors of said symptoms:
voice and facial analysis;
analysis of tremor and movement;
Levodopa and monoamine metabolites levels in the subcutaneous interstitial fluid of said patients;
   an algorithm being provided which provides the patient with the daily therapeutic scheme with the relative dosages and intermediate times of administration, calculated using said parameters as detectors.

The system for the management of oral therapy of Parkinson's disease includes:
an electronic bracelet, equipped with an accelerometer, a magnetometer and a triaxial gyroscope, used to collect continuous data on tremor and movement;
a camera to capture facial expressions, to be activated at the request of the patient;
a recorder to record the patient's voice to be activated at the patient's request;
a microdialysis pump connected to an analyzer unit and a subcutaneous needle designed to collect and analyze samples of interstitial fluid to determine the levels of levodopa and monoamine metabolites in said subcutaneous interstitial fluid;
a processing device, equipped with suitable software which, using a movement stabilization algorithm, designed to integrate movement, tremor, facial, vocal data and the levels of levodopa and monoamine metabolites in said subcutaneous interstitial fluid, provides information to the patient to modulate oral therapy, using data from the initial therapeutic scheme as feedback;
a display showing the updated oral therapy.

According to a preferred embodiment, the camera, the recorder, the processing device, the display and the relative software are integrated in a palmtop/smartphone.

In practice, the proposed invention modulates oral therapy using as feedback data from the initial therapeutic scheme, analysis of movement, tremor, facial, vocal, and levels of levodopa and monoamine metabolites in the subcutaneous interstitial fluid.

The advantage of this device is that, through multi-parametric monitoring (motor, tremor, vocal, facial, biochemical) it guarantees the patient to maintain an optimal motor performance (state of motor ON) continuously, providing the patient with a personalized therapeutic scheme, which varies according to the fluctuations of motor performance avoiding the states of motor OFF and dyskinesias.

Furthermore, the system is minimally invasive.

It will be immediately obvious that innumerable variations and modifications (for example relating to shape, dimensions, arrangements and parts with equivalent functionality) can be made to what is described, without departing from the scope of the invention, as appears from the attached claims.

It is understood that all attached claims form an integral part of the present description.

The present invention will be better described by a preferred embodiment, given by way of non-limiting example, with reference to the attached drawings, in which:

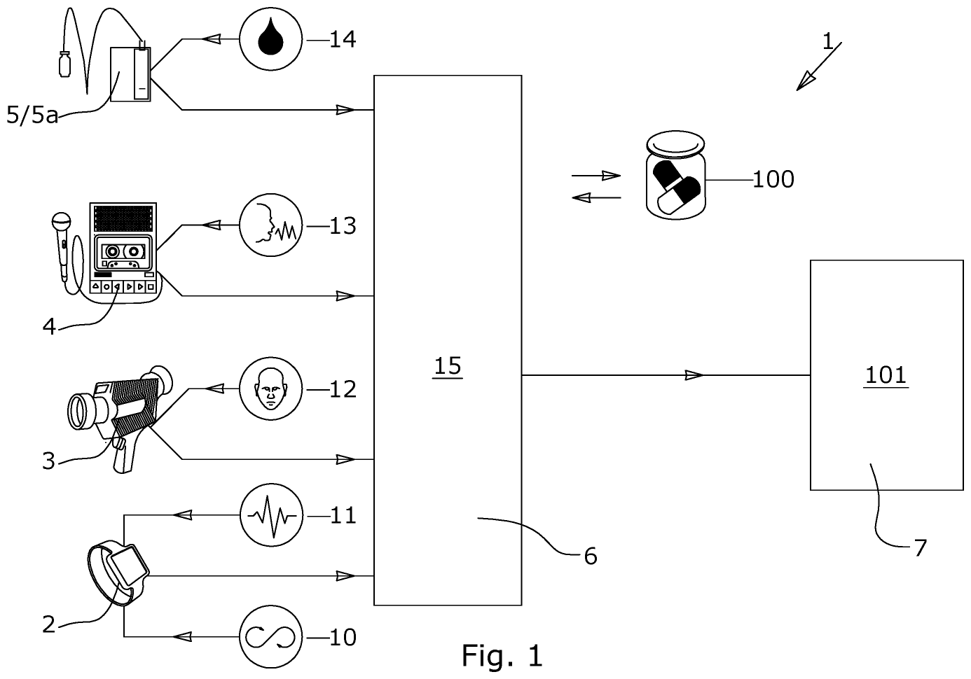

FIG. 1 shows a system for the management of oral therapy in Parkinson's disease, according to the invention;

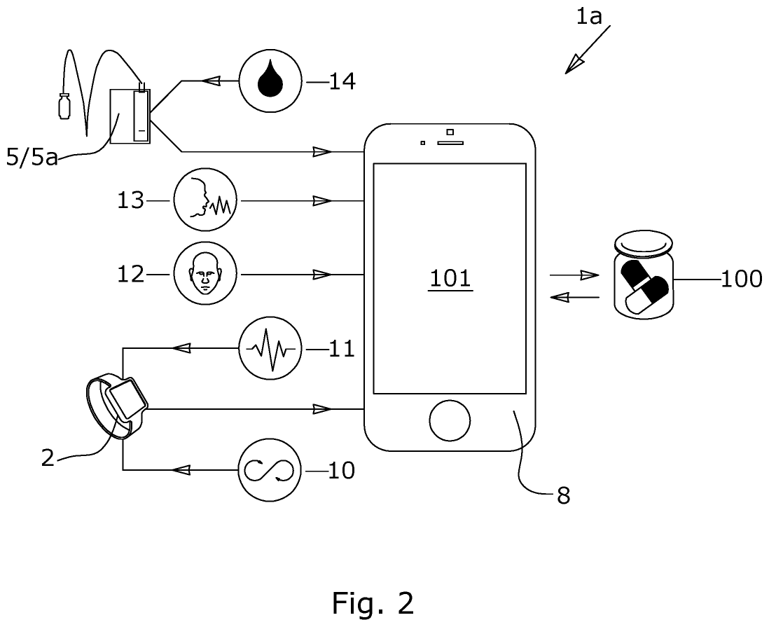

FIG. 2 shows a variant of the system according to the invention.

With reference to FIG. 1, the system (1) for oral therapy management support in Parkinson's disease of the invention includes:
an electronic bracelet (2), equipped with an accelerometer, a magnetometer and a triaxial gyroscope, used to continuously collect data on tremor and movement;
a camera (3) for recording facial expressions, to be activated at the request of the patient;
a recorder (4) to record the voice to be activated at the request of the patient;
a micro-dialysis pump (5) connected to an analyzer unit (5a) and with a subcutaneous needle for the collection of samples of interstitial fluid to determine the levels of levodopa and monoamine metabolites in said subcutaneous interstitial fluid;
a processing device (6), equipped with suitable software which, using a movement stabilization algorithm, integrates movement, tremor, facial, vocal data and the levels of levodopa and monoamine metabolites in said subcutaneous interstitial fluid, information to the patient to modulate oral therapy, using data from the initial therapeutic scheme as feedback;
a display (7) showing the updated oral therapy.

Starting from an initial therapy (100), established on the basis of the symptoms observed, the functioning of the system (1), to modulate the therapy according to the course of the disease, is divided into the following steps.

Step 1—The electronic bracelet (2) continuously records the patient's movement (10) and tremor (11) data.

Step 2—When the patient perceives a state of OFF or marked dyskinesias (DIS), he uses the camera (3) to record facial expressions (12) and the recorder (4) to record his voice (13). At the same time, the analyzer unit (5a) detects the levels of levodopa and monoamine metabolites (14) in the interstitial fluid samples taken from the micro-dialysis pump (5) through said subcutaneous needle.

Step 3—The processing device (6) receives from the electronic bracelet (2), the camera (3), the recorder (4) and the analyzer unit connected with the micro-dialysis pump (5), the combined movement data (10), tremor (11), facial images (12), voice (13) and levels of levodopa and monoamine metabolites (14) and, through the movement stabilization algorithm (15), determines the new daily therapeutic scheme (101) and shows it on the display (7).

According to a preferred embodiment (la), shown in FIG. 2, the camera (3), the recorder (4), the processing device (6), the display (7) and the related software are integrated in a PDA/smartphone (8), for which the electronic bracelet (2) and the analyzer unit (5a) connected to the micro-dialysis pump (5), send the analysis data of movement (10) and tremor (11) and levels of levodopa and monoamine metabolites (14) directly to the PDA/smartphone (8), which will show the new daily therapeutic scheme on its display (101).

The daily therapeutic scheme with the relative dosages and intermediate times of administration, will maintain a motor state of ON in a continuous and constant way throughout the day, without motor OFF or dyskinesias.

1. A method for managing an oral therapy for the treatment of Parkinson's disease, said method comprising determining a motor status of a patient affected by said Parkinson's disease, wherein said motor status can be in one of the following phases:
OFF phase, wherein Parkinson's symptoms, comprising one or more of rigidity, tremor and bradykinesia, emerge;
ON phase, wherein said symptoms markedly improve;
DYS phase, wherein involuntary movements emerge, called dyskinesias;
wherein said method uses as detectors of said symptoms the following parameters:
voice analysis;
face analysis;
tremor analysis;
movement analysis;
levodopa and metabolite levels of monoamines in the sub-cutaneous interstitial liquid of said patients;
providing to the patient a daily therapeutic scheme with its related administration dosages and inter-times, computed by using said parameters as detectors.

2. A system for managing an oral therapy for the treatment of Parkinson's disease, comprising:
an electronic bracelet, equipped with an accelerometer, a magnetometer and a gyroscope, three-axial, for collecting data continuously about tremor and movement;
a photo-camera to shoot face expressions, to be actuated upon request of a patient;
a recorder to record the patient voice, to be actuated upon request of the patient;
a micro-dialysis pump connected to an analysing unit and to a sub-cutaneous needle, adapted to collect and analyse samples of interstitial liquid to determine levodopa and metabolite levels of monoamines in said sub-cutaneous interstitial liquid;
a processing device, equipped with a suitable software which, using a movement stabilizing algorithm, adapted to integrate data about movement, tremor, face, voice and levodopa and metabolite levels of monoamines in said sub-cutaneous interstitial liquid, to provide the patient with information to modulate the oral therapy, using as feedback data about a starting therapeutic scheme;
a display on which the updated oral therapy is displayed.

3. The system for managing an oral therapy for the treatment of Parkinson's disease according to claim 2, wherein said photo-camera, said recorder, said processing device, said display and the related software are integrated in a handheld computer/smartphone, so that the electronic bracelet and the analysing unit send analysis data about movement, tremor and levodopa and metabolite levels of monoamines directly to said handheld computer/smartphone, said handheld computer/smartphone being equipped with a display adapted to display updated oral therapy.
